(19) United States
(12) Patent Application Publication
(10) Pub. No.: US 2026/0132435 A1
(43) Pub. Date: May 14, 2026

(54) METHOD FOR DEGRADING AND REUSING DISCARDED CELLULOSE PRODUCTS

(71) Applicant: JINJIANG ANRUN TEXTILE CO., LTD., Quanzhou (CN)

(72) Inventors: Honghui YIN, Quanzhou (CN); Jinbin XIAO, Quanzhou (CN)

(21) Appl. No.: 19/401,606

(22) Filed: Nov. 26, 2025

Related U.S. Application Data
(63) Continuation of application No. PCT/CN2024/132237, filed on Nov. 15, 2024.

(30) Foreign Application Priority Data
Dec. 5, 2023 (CN) 202311655245.3

(51) Int. Cl. C12P 19/04; C12N 9/42; C12P 39/00

(52) U.S. Cl. CPC C12P 19/04; C12N 9/2437; C12P 39/00; C12P 2201/00

(57) ABSTRACT

A method for degrading and reusing discarded cellulose products, including using recycled waste cellulose products and fermenting them with a mixed culture of Trichoderma viride, Trichoderma reesei, Aspergillus niger, and Trichoderma pseudokoningii to obtain crude cellulase products.

### CROSS-REFERENCE TO RELATED APPLICATIONS

This application is a continuation of International Application No. PCT/CN2024/132237, filed on Nov. 15, 2024, which claims priority to Chinese Patent Application No. 202311655245.3, filed with the China National Intellectual Property Administration on Dec. 5, 2023, both of which are hereby incorporated by reference in their entireties.

### TECHNICAL FIELD

The present disclosure relates to a method for degrading and reusing discarded cellulose products.

### BACKGROUND

Cellulose is a large molecular polysaccharide that is insoluble in organic solvents and water, and is widely present in plant cell walls in nature. Due to the huge source of cellulose in nature, people have directly extracted or extracted it through different chemical processes, and then prepared cellulose fibers with different crystal structures through spinning technology. With their good mechanical properties, moisture permeability, dyeing performance, and low cost, they have been widely used in clothing and home decor products. However, with the widespread application of cellulose fibers, the problem of waste disposal of cellulose products has become increasingly severe. Currently, the commonly used methods are incineration, landfill, etc., which pose corresponding hazards to environmental safety and have low utilization rates. The biodegradation of cellulose products refers to the process in which the products, after being discarded, rely on microorganisms in the natural environment such as bacteria, mold, and algae to lose their original form and undergo natural decomposition. But it is expected to take more than a year to fully degrade through natural degradation. At present, there are five main methods for evaluating the biodegradability of textile materials: activated sludge method, soil decomposition method, aerobic composting method, specific microbial or enzyme action method, and anaerobic test. Niu Jiantao et al. studied the biodegradation of washed cotton, linen, and viscose fibers by Aspergillus oryzae for 2 weeks. Infrared spectroscopy did not generate any new substances, but the internal structure of the fibers was damaged to some extent. Lin Yanping studied the high-temperature composting of cellulose products by crushing them and adding them to compost. After 3 months, the quality degradation rate of cellulose products was greater than 50%. There are also studies showing that recycling 1 kg of waste clothing can reduce 3.6 kg of carbon dioxide emissions and save 6000 L of industrial water.

Based on the above, a method for degrading and reusing discarded cellulose products is provided.

### SUMMARY

The purpose of the present disclosure is to provide a method for degradation and reuse of waste cellulose products that can improve the degradation efficiency of cellulose products and reduce costs.

In order to achieve the above objectives, the present disclosure adopts the following technical solution.

A method for degrading and reusing discarded cellulose products, including the following steps:
step A, pre-sorting and crushing recycled waste cellulose products to tear a fabric into fabric fragments;
step B, placing the fabric fragments in a H₂SO₄ solution with a mass fraction concentration of 2%-6% for 12-16 hours, a mass ratio of H₂SO₄ solution to the fabric fragments is 1-2:2-4;
step C, adding bran and/or soybean meal as a nitrogen source to the solution in step B, a mass ratio of the nitrogen source to the fabric fragments is 1-2:8-9; adding 20-60 mL of a nutrient solution per kilogram of the fabric fragments, mixing evenly to form a mixed material;
step D, adjusting a water content mass fraction of the mixed material to 55%-65%, pH to 4.5-6.5, and carrying out a sterilization treatment;
step E, adding a mixed culture with a 5%-10% mass fraction, and the mixed culture is a culture medium containing two or more of Trichoderma viride, Trichoderma reesei, Aspergillus niger, and Trichoderma pseudokoningii to the mixed material;
step F, performing an aerobic fermentation on the mixed material processed in step E, with a fermentation temperature of 20-35° C. and a fermentation time of 20-30 days; monitoring an enzymatic activity of cellulase, and when the enzymatic activity reaches its peak, air drying an enzyme koji to obtain crude cellulase product.

In some embodiments of the present disclosure, the waste cellulose products include cotton fibers, hemp fibers, and sugarcane fibers.

In some embodiments of the present disclosure, the nitrogen source is a mixture of bran and soybean meal, and a mass ratio of the bran to soybean meal is 2-4.

In some embodiments of the present disclosure, the nutrient solution includes 0.5-4 g/L of ammonium sulfate, 1-3 g/L of potassium dihydrogen phosphate, 1-3 g/L of magnesium sulfate, and remaining water.

In some embodiments of the present disclosure, a concentration of mycelium in the culture medium is controlled at 3-8 g/L.

In some embodiments of the present disclosure, the culture medium is prepared by adding 200 g of potatoes to 1000 mL of water, boiling for 30 minutes, filtering with gauze, and replenishing water to 1000 mL.

In some embodiments of the present disclosure, a cultivation time of the mixed culture is 45-50 hours and a cultivation temperature is 28-32° C.

In some embodiments of the present disclosure, a diameter of the fabric fragments is ≤10 cm or a length thereof is ≤10 cm.

In some embodiments of the present disclosure, a processing temperature in step B is room temperature and is treated in a sealed environment.

In some embodiments of the present disclosure, a sterilization temperature in step D is 121° C. and a sterilization time is 30 minutes.

After adopting the technical solution of the present disclosure, a dry dilute acid pretreatment method is used to pre modify some of the additives in the cellulose product and separate them from the cellulose material, thereby forming voids and gaps inside the cellulose product for later microbial degradation. The present disclosure adopts a mixed culture collaborative degradation method to compensate for the shortcomings of incomplete enzyme system and low enzyme production of a single strain, improve microbial degradation efficiency. The present disclosure adds nitrogen source auxiliary materials such as bran and soybean meal, and nutrient solution to fully reuse the cellulose raw material, producing crude cellulase. Crude cellulase can turn waste cellulose products into treasure, and the production cost of enzymes is lower than traditional methods. Its social and economic benefits will be obvious. The crude cellulase obtained by the present disclosure can be applied to the pretreatment process of straw cellulose, which can reduce the cost of using some cellulases.

### DESCRIPTION OF EMBODIMENTS

In order to better understand the technical solution of the present disclosure, a more detailed explanation will be provided below in combination with the embodiments.

### Example 1

Recycled waste cellulose products (taking cotton fiber products as an example, the same below) are sorted and crushed in advance, and the products are torn into fabric fragments with a diameter of ≤10 cm using a double axis shredder; adding dilute sulfuric acid with a mass fraction of 2%, with a mass ratio of 2:2 between the dilute sulfuric acid and the fiber products, and sealing at room temperature for 16 hours; adding a nitrogen source of bran and soybean meal, with a mass ratio of bran:soybean meal=2, and a mass ratio of an addition amount to a fabric dry basis being of 1:9, adding a nutrient solution 20 mL/kg to fabric dry basis, and mixing evenly to obtain a mixed material; adjusting a water content of the mixed material to 55%, and then adjusting a pH of the mixed material to 6.5. Adding a mixed culture with a mass fraction of 5% (Trichoderma viride:Trichoderma reesei:Aspergillus niger: Trichoderma pseudokoningii=1:2:1:1). A mycelium concentration of an inoculum is controlled at 5 g/L. After 30 days of aerobic fermentation (i.e. aerobic fermentation, using conventional technology in the biochemical field), a filter paper activity of the crude cellulase product reaches 200 IU/g dry yeast.

### Example 2

The recycled waste cellulose products are sorted and crushed in advance, and the products are torn into fragments with a diameter of ≤10 cm using the double axis shredder; adding dilute sulfuric acid with a mass fraction of 4%, with a mass ratio of 2:3 between the dilute sulfuric acid and the fiber products, and sealing at room temperature for 14 hours. Adding a nitrogen source of bran and soybean meal, with a mass ratio of bran to soybean meal being 3, and a ratio of 2:8 for the addition amount to the fabric dry basis. Adding the nutrient solution 20 mL/kg to fabric dry basis and mixing evenly to obtain a mixed material; adjusting the water content of the mixed material to 60%, and then adjusting the pH of the mixed material to 5.5. Adding a mixed culture with a mass fraction of 8% (Trichoderma viride:Trichoderma reesei:Aspergillus niger:Trichoderma pseudokoningii=1:1:1:2), controlling the mycelium concentration of the inoculum to 8 g/L, and fermenting the inoculum with oxygen at 30° C. for 25 days to produce the filter paper activity of the crude cellulase product to 220 IU/g dry yeast.

### Example 3

The recycled waste cellulose products are sorted and crushed in advance, and the products are torn into fragments with a diameter≤10 cm; adding dilute sulfuric acid with a mass fraction of 6%, with a mass ratio of 1:4 between the dilute sulfuric acid and the fiber products, and sealing at room temperature for 12 hours. Adding a nitrogen source of bran and soybean meal, with a mass ratio of 4 for bran to soybean meal, and 1:8 for the addition amount of the nitrogen source to the fabric dry basis. Adding 20 mL/kg of nutrient solution to dry basis and mixing evenly to obtain a mixed material; adjusting the water content of the mixed material to 65%, and then adjusting the pH of the mixed material to 4.5. Adding the mixed bacteria with a mass fraction of 10% (Trichoderma viride:Trichoderma reesei:Aspergillus niger:Trichoderma pseudokoningii=2:1:1:1), controlling the mycelium concentration of the inoculum to 3 g/L, and fermenting the inoculum with oxygen at 30° C. for 20 days. The filter paper activity of the crude cellulase product reached 210 IU/g dry yeast.

### COMPARATIVE EXAMPLE

In each comparative example, except for using a single strain for fermentation, the other conditions were the same as in Example 1.

A single strain of Trichoderma viride was used for fermentation, and the filter paper activity of the crude cellulase reached 45 IU/g dry yeast.

A single strain of Trichoderma reesei was used for fermentation, and the filter paper activity of the crude cellulase reached 70 IU/g dry yeast.

A single strain of Trichoderma pseudokoningii was used for fermentation, and the filter paper activity of the crude cellulase reached 170 IU/g dry yeast.

A single strain of Aspergillus niger was used for fermentation, and the filter paper activity of the crude cellulase reached 62 IU/g dry yeast.

In this application, IU refers to a temperature at 37° C., pH of 5.5, under a condition of 60 minutes of reaction, an amount of enzyme required to degrade filter paper and release 1 μmol of glucose per minute is one enzyme activity unit.

In various embodiments and comparative examples, Trichoderma viride, Trichoderma reesei, Aspergillus niger and Trichoderma pseudokoningii were cultured in a culture medium prepared as follows: 200 g of potatoes, 1000 ml of water, boiled for 30 minutes, filtered through four layers of gauze, supplemented with water to 1000 mL, cultured for 45-50 hours at a temperature of 28-32° C.

In this application: Trichoderma viride AS 3.3711, Trichoderma reesei ATCC 56764, Trichoderma lucidum 3.3002, Aspergillus niger CGMCC 3.316 can be purchased directly from the market.

Of course, the protection scope of the present disclosure is not limited to this embodiment, and anyone who makes similar changes to it can be regarded as not departing from the protection scope of the present disclosure.

The present disclosure can improve the degradation efficiency of cellulose products and reduce costs.

What is claimed is:

1. A method for degrading and reusing discarded cellulose products, comprising the following steps:
step A, pre-sorting and crushing recycled waste cellulose products to tear a fabric into fabric fragments;
step B, placing the fabric fragments in a H₂SO₄ solution with a mass fraction concentration of 2%-6% for 12-16 hours, a mass ratio of H₂SO₄ solution to the fabric fragments is 1-2:2-4;
step C, adding bran and/or soybean meal as a nitrogen source to the solution in step B, a mass ratio of the nitrogen source to the fabric fragments is 1-2:8-9; adding 20-60 mL of a nutrient solution per kilogram of the fabric fragments, mixing evenly to form a mixed material;
step D, adjusting a water content mass fraction of the mixed material to 55%-65%, pH to 4.5-6.5, and carrying out a sterilization treatment;
step E, adding a mixed culture with a 5%-10% mass fraction, and the mixed culture is a culture medium containing two or more of Trichoderma viride, Trichoderma reesei, Aspergillus niger, and Trichoderma pseudokoningii to the mixed material;
step F, performing an aerobic fermentation on the mixed material processed in step E, with a fermentation temperature of 20-35° C. and a fermentation time of 20-30 days; monitoring an enzymatic activity of cellulase, and when the enzymatic activity reaches its peak, air drying an enzyme koji to obtain crude cellulase product.

2. The method for degrading and reusing waste cellulose products according to claim 1, wherein the waste cellulose products comprise cotton fibers, hemp fibers, and sugarcane fibers.

3. The method for degrading and reusing waste cellulose products according to claim 2, wherein the nitrogen source is a mixture of bran and soybean meal, and a mass ratio of the bran to soybean meal is 2-4.

4. The method for degrading and reusing waste cellulose products according to claim 3, wherein the nutrient solution comprises 0.5-4 g/L of ammonium sulfate, 1-3 g/L of potassium dihydrogen phosphate, 1-3 g/L of magnesium sulfate, and remaining water.

5. The method for degrading and reusing waste cellulose products according to claim 4, wherein a concentration of mycelium in the culture medium is controlled at 3-8 g/L.

6. The method for degrading and reusing waste cellulose products according to claim 5, wherein the culture medium is prepared by adding 200 g of potatoes to 1000 ml of water, boiling for 30 minutes, filtering with gauze, and replenishing water to 1000 mL.

7. The method for degrading and reusing waste cellulose products according to claim 6, wherein a cultivation time of the mixed culture is 45-50 hours and a cultivation temperature is 28-32° C.

8. The method for degrading and reusing waste cellulose products according to claim 7, wherein a diameter of the fabric fragments is ≤10 cm or a length thereof is ≤10 cm.

9. The method for degrading and reusing waste cellulose products according to claim 8, wherein a processing temperature in step B is room temperature and is treated in a sealed environment.

10. The method for degrading and reusing waste cellulose products according to claim 9, wherein a sterilization temperature in step D is 121° C. and a sterilization time is 30 minutes.
